(19) United States
(12) Patent Application Publication
(10) Pub. No.: US 2021/0300975 A1
(43) Pub. Date: Sep. 30, 2021

(54) PROCESS FOR THE SEPARATION AND PURIFICATION OF PHYCOBILIPROTEINS

(71) Applicant: CONSIGLIO NAZIONALE DELLE RICERCHE, Roma (IT)

(72) Inventors: Rosaria LAUCERI, Cambiasca (VB) (IT); Giuseppe TORZILLO, Campi Bisenzio (F!) (IT); Graziella CHINI ZITTELLI, Borgo San Lorenzo (FI) (IT)

(73) Assignee: Consiglio Nazionale delle Ricerche, Roma (IT)

(21) Appl. No.: 17/057,831

(22) PCT Filed: Jun. 4, 2019
(86) PCT No.: PCT/IB2019/054619

§ 371 Date: Nov. 23, 2020

(30) Foreign Application Priority Data
Jun. 6, 2018 (IT) 102018000006062

(51) Int. Cl. C07K 14/405; C07K 1/16; C07K 1/34; C07K 1/36

(52) U.S. Cl. CPC C07K 14/405; C07K 1/36; C07K 1/34; C07K 1/16

(57) ABSTRACT

The present invention relates to a membrane chromatography process for selectively separating and purifying phycobiliproteins starting from aqueous biomasses of cyanobacteria and algae that, in a short time and at reduced cost, allows to obtain phycobiliproteins with a high degree of purity for use in pharmaceutical, cosmetic, or food compositions or as reagents for research.

### FIELD OF THE INVENTION

The present invention relates in general to the field of the purification of active ingredients from natural products, and more precisely it refers to a process of membrane chromatography for obtaining phycobiliproteins with a high degree of purity starting from cyanobacteria and/or algae biomasses.

### STATE OF THE ART

The phycobiliproteins are components with bright, highly fluorescent colors of the antenna complexes of the photosynthetic system of cyanobacteria and some algae, such as the algae belonging to the classes Rhodophyta, or red algae, and Cryptophyta, or cryptomonads. The phycobiliproteins are formed by a complex of proteins and linear tetrapyrrolic groups—which represent the chromophores in the complex—in which the tetrapyrrolic groups are covalently linked to the protein units. The most common phycobiliproteins are phycocyanin, allophycocyanin and phycoerythrin, the first two being blue, the third being bright and the third being fuchsia. These proteins are natural water-soluble pigments which, in addition to playing an important biological role in the collection of light in the organisms in which they are present, also represent products with high added value and various commercial applications. Due to their properties of pigments with fluorescence they are in fact used as natural colorants in the cosmetic field or as fluorescent probes (e.g. in flow cytometry or for immunological analysis), and as a coloring additive in the food field.

These proteins have also been recognized since long as having a high nutritional power and as bioactive molecules, hence their use as food (the two phycobiliproteins phycocyanin and allophycocyanin extracted from spirulina algae are approved for food consumption by EFSA in Europe and by FDA in the United States), and as a non-toxic photosensitizer in the photodynamic therapy (PDT) of tumors, while there are numerous studies on the potential use of these proteins as a drug based on the now proven antioxidant, anti-inflammatory, neuroprotective, antitumoral and immunomodulating properties (see for example [1] and [2]).

With such and so many applications it is clear the commercial value of these products: their value may however considerably vary depending on the degree of purity of the protein conjugate that on its turn varies with the type of application. For food applications, for instance, a degree of purity higher than 0.7 or “food grade” is required, for the cosmetic use the required degree of purity or “cosmetic grade” is higher than 2.0, higher than 3.9 the reactive grade and higher than 4.0 the analytic grade required to phycobiliproteins. In general, as also is in the following description, by “purity degree” of a phycobiliprotein the ratio is meant between the maximum absorbance value of the phycobiliprotein (around 540-570 nm for phycoerythrin, around 615-620 nm for phycocyanin, around 650 nm for allophycocyanin) and the absorbance value at 280 nm, which is related to the total amount of proteins in the product (see for instance references [3], [4]).

So far, many methods have been proposed to purify phycobiliproteins and the phycocyanin in particular. Usually, with these methods, a high degree of purity is achieved only after several purification steps, which generally include several steps of column chromatography of the biomass extracts that contain the product of interest, with a consequent yield reduction and a considerable increase of the manufacturing costs, which make impractical the exploitation of these methods on industrial scale. See for example [5], [6].

Alternatively, some simplified chromatographic purification procedures have been proposed, on paper more suitable for large-scale use; however, only a few of these protocols are able to avoid purification processes by column chromatography and among these, only those using extraction with a two-phases aqueous system, combined with ultrafiltration—a methodology that is also long and expensive—are successful to provide an analytical grade product [7].

In fact, it is known that the traditional packed column chromatography presents a series of disadvantages, such as the slowness of the separation by diffusion between the pores and the consumption of large volumes of solvents, which limit its application on a large scale, especially in the purification of large molecules. On the other hand, more recently, other types of chromatographic separation have been proposed for the purification of products, such as membrane chromatography, which have become increasingly important (see for example [8]), in particular for processing large volumes of dilute solutions, also in the purification of large biomolecules, such as nucleic acids, proteins, antibodies, or viruses. As a matter of fact, membrane chromatography has considerable advantages compared to column chromatography, from a reduced solute transport time to a lower volume of solvent required for the same recovered product. The bonding efficiency of the membranes towards the solutes is also generally independent from the speed of the supply flow to the membrane over a wide range, therefore very high flows can also be used, thus improving the process time. Finally, the devices are simpler and less expensive to be mass-produced. On the other hand, however, membrane chromatography is characterized by a low load capacity and by a not high resolution in the separation processes. In the literature, these disadvantages, in particular with respect to column chromatography, have been discussed and highlighted, for example in [9].

Furthermore, in this type of chromatography, the functionalization of the membrane with appropriate ligands can allow the exploitation of the most various chemical processes to achieve separation, such as for example affinity, ion exchange, or hydrophobic interactions. However, it was highlighted that different materials used to produce chromatography membranes have a certain intrinsic capacity to selectively interact with proteins, with consequences on the possibility of using this type of chromatographic separation for protein purification.

To date, therefore, as far as the Applicant is aware, efficient purification procedures suitable for use on a large scale are not known, which allow to reduce the production costs of the phycobiliproteins while ensuring acceptable levels of purity. This effectively limits the massive use of these chromoproteins in the various sectors of commercial application, despite the fact that their high nutraceutical and medical value, or even more simply the effectiveness of natural dye, is now acknowledged.

### SUMMARY OF THE INVENTION

Now the Applicant has found that it is possible to obtain single phycobiliproteins with a high degree of purity, in particular phycocyanin, allophycocyanin or phycoerythrin, starting from cyanobacterial and algal biomasses by membrane chromatography treatment.

This method, applied directly to raw aqueous extracts containing phycobiliprotein mixtures, has made it possible to obtain the separation of the products of interest with a high degree of purity, in a simple way and at low costs, as required for commercial uses in the pharmaceutical, food and cosmetic fields, as well as for the application in analysis and research.

The use of column chromatography or of other expensive and lengthy procedures, such as ultrafiltration, is also avoided thanks to the present process.

Subject of this invention is therefore a process for the separation and purification of phycobiliproteins from raw extracts of cyanobacteria and/or algae containing them, comprising at least a cycle of membrane chromatography purification, as defined in the first of the appended claims.

Other important features of the process according to the present invention are reported in the following detailed description.

### BRIEF DESCRIPTION OF THE FIGURES

FIG. 1—Absorption spectrum of purified phycocyanin obtained as described in the following Example 1.

FIG. 2—Absorption spectrum of the purified allophycocyanin obtained as described in the following Example 5.

FIG. 3—Absorption spectrum of purified B-phycoerythrin obtained as described in the following Example 8.

### DETAILED DESCRIPTION OF THE INVENTION

In accordance with the invention, the process proposed herein is a simplified process for the selective separation and purification of phycobiliproteins, in particular phycocyanin, allophycocyanin and phycoerythrin, from crude extracts of cyanobacteria and/or algae that contain mixtures of such phycobiliproteins, without the use of column chromatography or ultrafiltration or other long and expensive techniques. The inventors have in fact surprisingly found that it is possible to carry out an effective separation and purification of the phycobiliproteins present in a saline aqueous extract by passing through a microfiltration membrane, exploiting the selective and reversible bonding of the different phycobiliproteins with the membrane, without using long processes of elution with solvents as it is when using column chromatography. In other words, this is a membrane chromatography separation, wherein the microfiltration membrane represents the stationary phase and the aqueous saline solution represents the mobile phase of the chromatographic method.

In a preferred aspect of this invention, the starting raw extracts are cyanobacteria extracts or are algae extracts, for example selected from extracts of Arthrospira platensis (Spirulina) and extracts of Porphyridium cruentum. Preferably, the starting extracts are extracts of Arthrospira platensis. Furthermore, in the present process, extracts from fresh biomass or extracts from dehydrated biomass can be used, for example from freeze-dried biomass in powder form. According to a preferred embodiment of the process of this invention, the starting crude extracts were obtained by suspension in aqueous solution of a biomass of cyanobacteria and/or freeze-dried algae, subsequent centrifugation and collection of the so-formed supernatant. The suspension in aqueous solution of the freeze-dried biomass is preferably kept at rest, for example at 4° C. for 24 hours, before being subjected to centrifugation. The starting suspension can be for example a suspension in water or in an aqueous solution of a salt or of a buffer.

In a particular aspect of this process, the suspension from biomasses of cyanobacteria, algae, or from biomasses containing cyanobacteria together with algae, can be advantageously subjected to an extractive treatment before purification on the membrane, in order to break up the cellular walls of the organisms and free the proteins contained inside them, for a higher yield of the desired products. Any technician with ordinary skills in the art can effortlessly identify a treatment method suitable to this purpose, such as for example ultrasonication, freeze-drying, freezing/thawing cycles or enzymatic treatments.

In a particular embodiment of the invention, the crude extracts from dry freeze-dried biomass, obtained by suspension of the biomass in a saline solution and subsequent centrifugation, are directly subjected to membrane chromatography separation/purification. In another particular embodiment of the invention, the crude extracts from fresh, wet biomass are obtained thanks to several cycles of freezing/thawing of the suspension of the biomass in saline solution and subsequent centrifugation thereof, before being subjected to separation/purification by membrane chromatography.

The present process is in any case applicable to any cyanobacterium and/or algae, on crude extracts obtained in any way.

The present process of separation and purification of phycobiliproteins from crude extracts of cyanobacteria and/or algae containing them is based on membrane chromatography of the above said extracts, and comprises at least one purification cycle comprising:

i) at least one passage of a saline aqueous solution of the crude extracts on a hydrophilic porous membrane, having a low protein binding power, in which the aqueous saline solution of the crude phycobiliprotein extracts is an aqueous solution of a salt S having a concentration [S]₁;

ii) desorption of the retentate bound to the membrane with a solvent selected from the group consisting of water, an aqueous solution of said salt S at a concentration [S]₂<[S]₁, and an aqueous solution of a salt S′ that is a stronger chaotropic agent than the salt S.

In the above said process the hydrophilic porous membrane is the stationary phase of the chromatographic method, while the aqueous saline solution of the phycobiliproteins extracts in step i) represents a first mobile phase, which determines the interaction of phycobiliproteins with the membrane, and the solvent in step ii) of the process is a second mobile phase.

In the present invention, the expression “a salt S′ that is a stronger chaotropic agent than the salt S” means a salt S′ having a greater ability than the salt S to break the hydrophobic bonds and the hydrogen bonds that the proteins form with the membrane.

The salt S is preferably selected from ammonium sulfate and sodium sulfate; optimal results in terms of yield, separation and degree of purity are obtained in this process with ammonium sulfate. The aqueous solution of the salt S′ is preferably selected from between aqueous solution of sodium chloride and saline phosphate buffer. Any technician with ordinary skills in the art can without efforts select any other aqueous solutions of salts S′ of possible use in step ii) of the present process, among the solutions of salts able to favor the detachment of proteins from the membrane.

The hydrophilic porous membrane of possible use in the present process can be for example a flat, hollow, capillary or tubular membrane of hydrophilic material, selected for example between polycarbonate and PVDF (polyvinylidene fluoride); preferably the membrane material is PVDF. There are several commercial microfiltration membranes of this type, which can be used successfully in the present process.

In the context of this invention, by “membrane” in the purification step i) is meant a single membrane or a plurality of membranes, for example more membranes stacked one on top of the other.

In a preferred aspect of the process of the invention, before the purification step i) of the extracts on the membrane, this is previously subjected to a conditioning treatment with a saline aqueous solution of the salt S at the same concentration [S]₁ used in the subsequent step.

According to a preferred embodiment, which allows to obtain particularly high purity degrees of the separated phycobiliprotein, for example a purity of analytical grade ≥4.0, the process of the invention further comprises, before the purification step i) on the membrane and upstream of the possible conditioning of the membrane described above, a cleaning step on the membrane of the extracts with an aqueous solution of salt S at concentration [S]₀<[S]₁, wherein [S]₁ is the concentration of the salt S in the aqueous solution of the phycobiliproteins to be purified. This aqueous solution of the salt S at concentration [S₀] is a third mobile phase wherein the hydrophilic membrane always represents the stationary phase of a chromatographic method. This preliminary chromatographic cycle with a saline solution at lower concentration allows minimizing the interaction between the membrane and the phycobiliproteins that will pass therefore in the permeate, while on the membrane various impurities remain. The membrane used in this phase may also be of the same type as that used in the at least one subsequent purification step i).

In a preferred aspect of the invention, the process is carried out by replacing the hydrophilic porous membrane after the cleaning step, when present, using in all the other chromatographic cycles of the process the same hydrophilic porous membrane as defined above.

In order to recover more than one different phycobiliprotein from the same biomass that contains several of these proteins, the present process can comprise more purification cycles carried out consecutively with equal or increasing concentrations of saline aqueous solution of the salt S by passage on a membrane of the permeate obtained from the previous cycle until the separation of the desired phycobiliprotein, for example after n passages, at the passage n+1 a concentration [S]ₙ₊₁≥[S]ₙ has to be used.

In a preferred aspect of the process of the invention, before each cycle n of purification i) of the extracts on the membrane, this is previously subjected to a conditioning treatment with a saline aqueous solution of the salt S at the same concentration [S]ₙ used in the subsequent step.

According to a preferred embodiment of the present process, each purification cycle can be repeated more than once at the same salt concentration S until the phycobiliprotein of the desired degree of purity is obtained.

In the context of the present invention, by “high degree of purity” a degree of purity is meant that is ≥0.7, corresponding to the degree of purity required of the phycobiliproteins for food use, where the degree of purity is defined as described above. With the present process it is however possible to obtain the products of interest, as demonstrated in the experimental part that follows, even with much higher degrees of purity, and in particular ≥2.0, the degree of purity required for cosmetic use; ≥3.5, degree of purity required for the application of phycobiliproteins in scientific research, for example as fluorescent markers; and ≥4.0, degree of purity required for the medical-pharmaceutical use of phycobiliproteins, for example as antiviral, antioxidant, antibacterial, anti-inflammatory or tracer agents.

The main advantage of the process of the invention lies in the possibility of obtaining phycobiliproteins with a high degree of purity in a simple and rapid manner; on a laboratory scale, the inventors have carried out and completed each purification cycle in just 3-4 minutes.

Furthermore, the present process allows obtaining high degrees of purity of the phycobiliproteins, suitable for the various applications, from food to cosmetics, to pharmaceuticals, to analytics.

A further advantage of the present process lies in the fact that all the reagents and devices used, including the microfiltration membranes used as chromatographic device, are commercially available at low cost, or in any case at a limited cost. The unit cost of the finished product will therefore also be proportionately lower.

A further advantage of the present process is its flexibility and re-adaptation depending on the phycobiliprotein to be recovered and of the related degree of purity desired. On the basis of these desiderata, the number of purification cycles by membrane chromatography, the related concentrations of salt S to be used and the possible preliminary passage of cleaning may be defined from time to time, so as to modulate the result of the process and obtain the desired product, having the desired purity, optimizing the yield.

The following Examples are reported for illustrative and not limitative purposes of the present invention.

### Example 1

Purification of Phycocyanin from Aqueous Extracts of Arthrospira platensis (Spirulina) Biomass

A crude phycobiliprotein extract was obtained by suspending the freeze-dried A. platensis biomass in an aqueous solution of 100 mM NaCl (140 mg of Spirulina in 11 mL of solution). The suspension was kept at 4° C. for 24 hours, then centrifuged for 45 minutes (12000 rcf, T=10° C.). The supernatant containing phycocyanin (PC) and allophycocyanin (APC) (hereinafter “crude extract”) was recovered and stored at 4° C.

The chromatographic purification process on the crude extract was performed by using a vacuum glass device for microfiltration and a PVDF microfiltration membrane, carrying out two consecutive chromatographic cycles. The second cycle has allowed to obtain phycocyanin with a degree of purity of analytical grade.

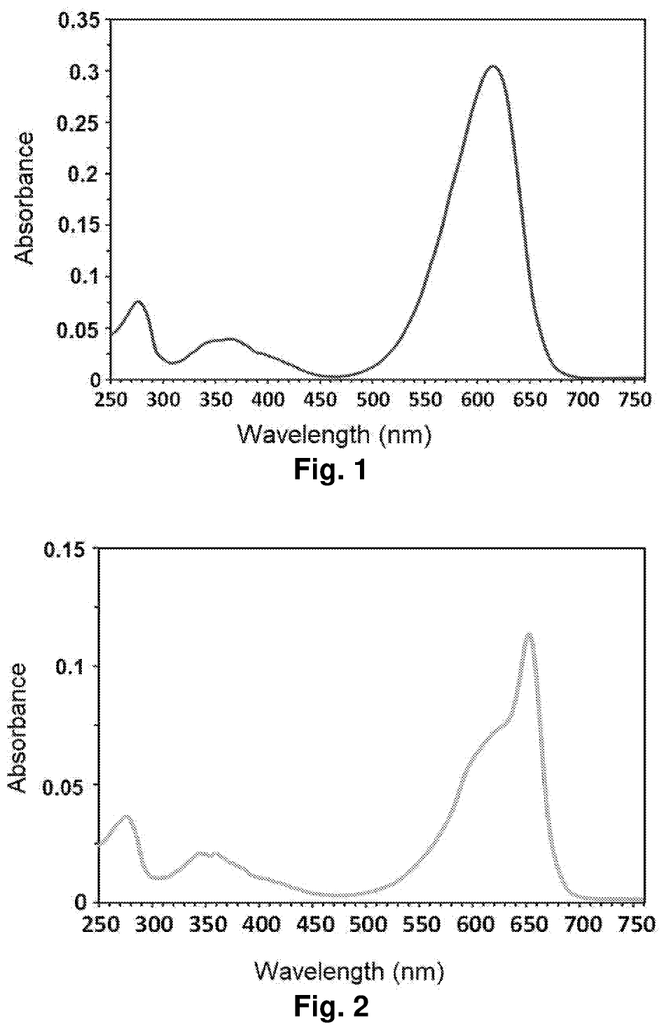

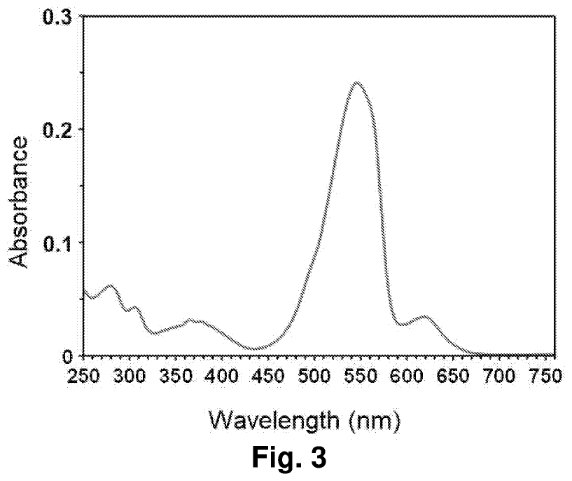

More specifically, a glass vacuum flask for microfiltration was assembled with a hydrophilic PVDF membrane (Durapore®, with an average pore size of 0.45 μm, diameter of 47 mm, thickness 125.0 μm, code HVLP 04700). Using this chromatographic device, the following two steps were conducted:
chromatographic cleaning cycle: the membrane was washed with 100 mL of deionized water and then conditioned with 5 mL ammonium sulfate 0.6 M. A volume of 100 mM NaCl aqueous solution was added to a 0.8 mL aliquot of crude extract and of ammonium sulfate 3 M so as to reach a final volume of extract in ammonium sulfate 0.6 M of 5 mL, with a total content of phycobiliproteins (PC+APC) of about 1 mg. The solution was loaded on the membrane and filtered; the permeate was collected in a 100 mL clean tailed flask. 5 mL of ammonium sulfate 0.6 M were then loaded onto the membrane, filtered and collected in the flask containing the permeate, to maximize the recovery of phycobiliproteins. No blue compound was adsorbed on the membrane at the ammonium sulfate concentration of 0.6 M. The volume of the solution was carefully measured and, if necessary, it was brought back to 10 mL by using an ammonium sulfate solution 0.6 M;
purification cycle: to the ammonium sulfate permeate 0.6 M (10 mL) obtained as described above in the cleaning cycle i), an aqueous solution of ammonium sulfate 3 M was added so as to obtain a phycobiliprotein solution having a concentration of 1.110 M ammonium sulfate. This solution was then loaded onto a new PVDF membrane, using the usual procedure: the membrane was washed with 100 mL of deionized water and then conditioned with 5 mL of an ammonium sulfate solution having the same ammonium sulfate concentration of the phycobiliprotein solution, i.e. 1.110 M. The solution of phycobiliprotein and ammonium sulfate 1.110 M was then loaded onto the membrane and filtered. The retentate on the membrane was washed with 10 mL of ammonium sulfate solution 1.110 M, then desorbed with 10 mL of NaCl solution 100 mM and recovered in a 100 mL clean tailed flask. The absorbance spectrum of the purified PC thus obtained is shown in FIG. 1.

The phycocyanin obtained resulted, in particular, to have a purity degree of 4.20, while the purification yield with respect to the phycocyanin content in the portion of crude extract subjected to purification was 67%.

### Example 2

Purification of Phycocyanin from Aqueous Extracts of Arthrospira platensis (Spirulina) Biomass

The crude extract of phycobiliproteins was obtained from fresh biomass of A. platensis. An aliquot of cultivation of biomass in water (40 mL) was centrifuged for 10 min (12,000 rcf, temperature=10° C.) and the supernatant eliminated. The cyanobacteria pellet was suspended and washed with 18 MO Milli-Q pure water, then centrifuged for 10 min (12,000 rcf, temperature 10° C.) and the supernatant eliminated. The pellet was resuspended in 10 mL of a NaCl aqueous solution 100 mM and subjected to three freeze-thaw cycles (in each cycle the suspension was kept at −20° C. for 2.5 hours and then thawed in a water bath at 20° C.). After adding further 10 mL of NaCl aqueous solution 100 mM (20 mL in total) the suspension was sonicated four times for 60 s (power 75%, pulse 60%, sonicating tip S2, Hielscher Ultrasonic Processor UP200S, 200 W, 24 kHz) in a bath of water and ice, with an interval of 60 s between a sonication cycle and the subsequent one. The suspension was finally centrifuged for 45 minutes (12,000 rcf, temperature=10° C.) and the supernatant containing phycocyanin (PC) and allophycocyanin (APC) (crude extract) was recovered and stored at 4° C.

The purification process on the crude extract was performed using a glass vacuum device for microfiltration and a PVDF microfiltration membrane, carrying out three consecutive chromatographic cycles on the membrane. The third cycle has allowed to obtain phycocyanin with a degree of purity of analytical grade.

More specifically, a glass vacuum flask for microfiltration was assembled with a hydrophilic PVDF membrane (Durapore®, with an average pore size of 0.45 μm, a diameter of 47 mm, HVLP code 04700). Using this device, the following two steps were carried out:
cleaning cycle: the membrane was washed with 100 mL of deionized water and then conditioned with 5 mL of ammonium sulfate 0.6 M. A volume of NaCl aqueous solution 100 mM was added to a 2.0 mL aliquot of crude extract and of 3 M ammonium sulfate so as to reach a final volume of extract in ammonium sulfate 0.6 M of 3 mL, with a total content of phycobiliproteins (PC+APC) of about 1 mg. The solution was loaded on the membrane and filtered; the permeate was collected in a 100 mL clean tailed flask. 3 mL of ammonium sulfate 0.6 M were then loaded onto the membrane, filtered and collected in the flask containing the permeate, to maximize the recovery of phycobiliproteins. No blue compound was adsorbed on the membrane at the ammonium sulfate concentration of 0.6 M. The volume of the solution was carefully measured and, if necessary, it was brought back to 6 mL using an ammonium sulfate solution 0.6 M;
purification cycle: to the ammonium sulfate permeate 0.6 M (6 mL) obtained as described above in the cleaning cycle i), an aqueous ammonium sulfate solution 3 M was added so as to obtain a phycobiliprotein solution having a concentration of ammonium sulfate 1.110 M. This solution was then loaded onto a new PVDF membrane, using the usual procedure: the membrane was washed with 100 mL of deionized water and then conditioned with 5 mL of an ammonium sulfate solution having the same ammonium sulfate concentration of the phycobiliprotein solution, i.e. 1,110 M. The phycobiliprotein solution in ammonium sulfate 1.110 M was then loaded onto the membrane and filtered. The retentate bound on the membrane was washed with 10 mL of ammonium sulfate solution 1.110 M, then desorbed with 5 mL of NaCl solution 100 mM and recovered in a 100 mL clean tailed flask. The spectrophotometric analysis of the product showed a purity level of phycocyanin lower than the analytical degree. A further membrane chromatography purification cycle was therefore performed on the recovered phycocyanin solution. In this step the volume of solution was carefully measured and an ammonium sulfate solution 3 M was added so as to obtain a phycocyanin solution having an ammonium sulfate concentration of 1.110 M, which was then loaded onto the membrane and filtered. The retentate bound onto the membrane was washed with 10 mL of ammonium sulfate solution 1.110 M, then desorbed with 5 mL of NaCl aqueous solution 100 mM and recovered in a 100 mL clean tailed flask. The spectrophotometric analysis showed an analytical grade purity for the phycocyanin thus obtained. More specifically, the phycocyanin obtained was found to have a purity degree of 4.0, while the purification yield with respect to the phycocyanin content in the portion of crude extract subjected to purification was of 60.0%.

### Example 3

Purification of Phycocyanin from Aqueous Extracts of Arthrospira platensis (Spirulina) Biomass

A crude extract of phycobiliproteins was obtained as described above in Example 1. The purification process on this crude extract was performed using a syringe for microfiltration and a filter for syringe with a membrane in hydrophilic PVDF (Durapore®, with an average pore size of 0.45 μm, 33 mm diameter, code SLHV033RS). Using this device, the following two cycles of chromatographic purification were carried out:
cleaning cycle: the filter was washed with 5 mL of deionized water and then conditioned with 3 mL of ammonium sulfate 0.6 M, then it was used to filter a 2 mL aliquot of crude extract in ammonium sulfate 0.6 M with a content total phycobiliproteins (PC+APC) of about 0.31 mg. The solution was filtered and the permeate was collected in a 10 mL glass bottle. The filter was then washed with 1 mL of ammonium sulfate solution 0.6 M, collecting the solution in the same bottle containing the permeate to maximize the recovery of phycobiliproteins. No blue compound adsorbed on the filter at the ammonium sulfate concentration of 0.6 M was observed. The volume of the solution was then carefully measured and, if necessary, it was brought back to 3 mL using an ammonium sulfate solution 0.6 M;
purification cycle: to the permeate in ammonium sulfate 0.6 M (3 mL) obtained as described above in the cleaning cycle, a volume of aqueous solution of NaCl 100 mM and ammonium sulfate 3 M was added so as to reach a final volume of extract in ammonium sulfate 1.110 M of 5 mL. In order to obtain analytical grade phycocyanin, this solution was loaded onto a new filter for syringe with a PVDF membrane, following then the usual procedure: the filter was washed with 5 mL of deionized water and then conditioned with 3 mL of an aqueous ammonium sulfate solution having the same ammonium sulfate concentration of the phycobiliprotein solution, i.e. 1.110 M. The ammonium sulfate phycobiliprotein solution 1.110 M was filtered and the retentate on the membrane was washed with 3 mL of ammonium sulfate solution 1.110 M. The retentate was finally recovered with 3 mL of aqueous solution of NaCl 100 mM.

The phycocyanin obtained as described above resulted, in particular, to have a purity grade of 4.0, while the purification yield with respect to the phycocyanin content in the crude extract aliquot subjected to purification was 63.0%.

### Example 4

Purification of Phycocyanin from Aqueous Extracts of Arthrospira platensis (Spirulina) Biomass

A crude extract of phycobiliproteins was obtained by suspending the biomass of A. platensis lyophilized in an aqueous solution of ammonium sulfate 0.6 M (100 mg of Spirulina in 15 mL of solution). The suspension was kept at 4° C. for 24 hours, then centrifuged for 45 minutes (12,000 rcf, temperature=10° C.). The supernatant containing phycocyanin (PC) and allophycocyanin (APC) (hereinafter “crude extract”) was recovered and stored at 4° C.

The purification process on the crude extract was performed using a glass vacuum device for microfiltration and a PVDF microfiltration membrane, carrying out two consecutive chromatographic cycles on membrane. The second cycle has allowed to obtain phycocyanin with a degree of purity of analytical grade.

More specifically, a glass vacuum flask for microfiltration was assembled with a hydrophilic PVDF membrane (Durapore®, with an average pore size of 0.45 μm, a diameter of 47 mm, HVLP code 04700). Using this device, the following two steps were carried out:
cleaning cycle: the membrane was washed with 100 mL of deionized water and then conditioned with 5 mL of ammonium sulfate 0.6 M. A 1.5 mL aliquot of crude extract in ammonium sulfate solution 0.6 M, with a total phycobiliprotein content (PC+APC) of about 1 mg, was loaded on the membrane and filtered; the permeate was collected in a 100 mL clean tailed flask. 5 mL of ammonium sulfate 0.6 M were then loaded onto the membrane, filtered and collected in the flask containing the permeate, to maximize the recovery of phycobiliproteins. No blue compound was adsorbed on the membrane at the ammonium sulfate concentration of 0.6 M. The volume of the solution was carefully measured and, if necessary, it was brought back to 10 mL of final volume, using an ammonium sulfate solution 0.6 M;
purification cycle: to the ammonium sulfate permeate 0.6 M (10 mL) obtained as described above in the cleaning cycle, an aqueous ammonium sulfate solution 3 M was added so as to obtain a phycobiliprotein solution having a concentration of ammonium sulfate 1.110 M. This solution was then loaded onto a new PVDF membrane, using the usual procedure: the membrane was washed with 100 mL of deionized water and then conditioned with 5 mL of an ammonium sulfate solution having the same ammonium sulfate concentration of the phycobiliprotein solution, i.e. 1.110 M. The solution of phycobiliprotein and ammonium sulfate 1.110 M was then loaded onto the membrane and filtered. The retentate on the membrane was washed with 10 mL of ammonium sulfate solution 1.110 M, then desorbed with 10 mL of NaCl solution 100 mM and recovered in a 100 mL clean tailed flask.

The phycocyanin obtained resulted, in particular, to have a purity grade of 4.05, while the purification yield compared to the phycocyanin content in the crude extract aliquot subjected to purification was 60.0%.

### Example 5

Purification of Phycocyanin and Allophycocyanin from Aqueous Extracts of Arthrospira platensis (Spirulina) Biomass

A crude extract of phycobiliproteins was obtained as described above in Example 1. The purification process on the crude extract was then carried out using a glass vacuum device for microfiltration and a microfiltration membrane in hydrophilic PVDF (Durapore®, with average pore size of 0.45 μm, 47 mm diameter, HVLP code 04700). No preliminary chromatographic purification cycle was carried out, operating as follows:
first purification cycle: the membrane was washed with 100 mL of deionized water and then conditioned with 5 mL of aqueous ammonium sulfate solution 1.10 M. A 48 mL aliquot of crude extract in aqueous ammonium sulfate 1.10 M solution, with a total phycobiliprotein content of about 9.7 mg, was loaded on the membrane and filtered; the permeate was collected in a clean 250 mL tailed flask. The retentate (PC) on the membrane was washed with 10 mL of ammonium sulfate 1.10 M, then desorbed with 10 mL of NaCl 100 mM, recovered in a clean 100 mL tailed flask and its degree of purity was spectrophotometrically determined. The permeate was repeatedly (6 times in total) loaded onto the membrane to recover all the phycocyanin still present in the solution. Between one cycle and the subsequent one, the membrane was cleaned with 100 mL of deionized water and then conditioned with 5 mL of ammonium sulfate solution 1.10 M.
second purification cycle: to remove from the permeate containing allophycocyanin (APC), (obtained at the end of the first purification cycle described above) the small amount of PC still present, an aqueous ammonium sulfate solution 3 M was added up to a final concentration of 1.30 M, and the solution was loaded onto the previously cleaned membrane with 100 mL of deionized water and conditioned with 5 mL of ammonium sulfate solution 1.30 M, then it was filtered. The retentate bound onto the membrane was removed by cleaning the membrane with 100 mL of deionized water. The membrane was conditioned with 5 mL of ammonium sulfate solution 1.3 M and the permeate loaded and filtered again to eliminate any PC still present in the solution.
third purification cycle: an ammonium sulfate 3 M aqueous solution was added to the permeate obtained from the second purification cycle up to a final concentration of 1.50 M and the solution was loaded onto the membrane previously cleaned with 100 mL of deionized water and conditioned with 5 mL of ammonium sulfate solution 1.50 M, then it was filtered. The retentate (APC) on the membrane was washed with 10 mL of ammonium sulfate solution 1.50 M, then desorbed with 10 mL of aqueous solution of 100 mM NaCl and recovered in a 100 mL clean tailed flask. The degree of purity of APC was spectrophotometrically determined, from the absorption spectrum of FIG. 2.

The following Table 1 shows the degree of purity and the yield of product with respect to the content of phycocyanin and of allophycocyanin in the aliquot of crude extract subjected to purification.

TABLE 1

| Purification | No. of | Phycobili- | Degree | | Yield |
|---|---|---|---|---|---|
| cycle, AS | purification | protein of | of | Yield | (%) |
| concentration | step | interest | purity | (%) | (P ≥ 3.5) |
| I cycle, | 1 | PC | 3.6 | 17.1 | 63.8 |
| 1.10 M | | | | | |
| I cycle, | 2 | PC | 3.9 | 18.7 | |
| 1.10 M | | | | | |
| I cycle, | 3 | PC | 4.0 | 17.3 | |
| 1.10 M | | | | | |
| I cycle, | 4 | PC | 3.7 | 10.7 | |
| 1.10 M | | | | | |
| I cycle, | 5 | PC | 2.6 | 3.1 | |
| 1.10 M | | | | | |
| I cycle, | 6 | PC | 1.6 | <1 | |
| 1.10 M | | | | | |
| II cycle, | 1 | PC | — | — | |
| 1.30 M | | | | | |
| II cycle, | 2 | PC | — | — | |
| 1.30 M | | | | | |
| III cycle, | 1 | APC | 3.4 | 9.2 | |
| 1.50 M | | | | | |

### Example 6

Purification of Phycocyanin from Aqueous Extracts of Arthrospira platensis (Spirulina) Biomass—Obtaining Phycocyanin with Food/Cosmetic Purity

A crude extract of phycobiliproteins was obtained as described above in Example 1. The purification process was then performed using a glass vacuum device for microfiltration assembled with a hydrophilic PVDF membrane (Durapore®, average pore size 0.45 μm, diameter 47 mm, code HVLP 04700). The cleaning chromatographic cycle was not carried out, while only one purification cycle was carried out, as follows:
purification cycle: the membrane was washed with 100 mL of deionized water and then conditioned with 5 mL of aqueous ammonium sulfate solution 1.65 M. A 10 mL aliquot of crude extract in aqueous ammonium sulfate 1.65 M solution, with total content of phycobiliproteins equal to about 1.7 mg, was loaded on the membrane and filtered. The retentate bound onto the membrane was washed with 10 mL of ammonium sulfate solution 1.65 M, then desorbed with 10 mL of NaCl aqueous solution 100 mM, recovered in a 100 mL clean tailed flask and its degree of purity was spectrophotometrically determined.

The purified phycocyanin thus obtained has revealed a degree of purity equal to 2.5, and a yield with respect to the content of phycocyanin in the aliquot of crude extract subjected to purification of 90.0%.

### Example 7

Purification of Phycocyanin from Aqueous Extracts of Arthrospira platensis (Spirulina) Biomass on a Polycarbonate Membrane

A crude extract of phycobiliproteins was obtained as described above in Example 1. The chromatographic purification process was then carried out using a glass vacuum device for microfiltration assembled with a hydrophilic polycarbonate membrane (Whatman Nuclepore, average pore size 1.0 μm, diameter 47 mm, thickness 10 μm, code 111110); a chromatographic cycle of cleaning and a chromatographic cycle of purification were carried out, as described in Example 1. In particular we proceeded as follows:
cleaning cycle: the membrane was washed with 100 mL of deionized water and then conditioned with 5 mL of aqueous ammonium sulfate solution 0.6 M. At a rate of 0.75 mL of crude extract with a total phycobiliprotein content (PC+APC) equal to about 1 mg, a volume of aqueous solution of NaCl 100 mM and ammonium sulfate 3 M was added to give a final volume of extract in ammonium sulfate 0.6 M of 6 mL. The solution was loaded on the membrane and filtered; the permeate was collected in a 100 mL clean tailed flask. No blue compound was adsorbed on the membrane at the concentration of ammonium sulfate 0.6 M. The volume of the solution was carefully measured and, if necessary, it was brought back to 6 mL using an aqueous solution of ammonium sulfate 0.6 M;
purification cycle: to the permeate (obtained in the cleaning step described above) an aqueous solution of ammonium sulfate 3 M was added so as to obtain a phycobiliprotein solution having a concentration of ammonium sulfate 1.110 M. The solution was loaded onto a new polycarbonate membrane, using the usual procedure: the membrane was washed with 100 mL of deionized water and then conditioned with 5 mL of a solution of ammonium sulfate having the same concentration in ammonium sulfate of the phycobiliprotein solution (1.110 M). The phycobiliprotein solution in ammonium sulfate 1.110 M was loaded onto the membrane and filtered. The retentate bound onto the membrane was washed with 10 mL of an aqueous solution of ammonium sulfate 1.110 M, then desorbed with 10 mL of a 100 mM NaCl aqueous solution, recovered in a 100 mL clean tailed flask and its degree of purity was determined by spectrophotometry.

Phycocyanin was obtained with a purity degree of 2.44, and with a yield with respect to the phycocyanin content in the crude extract aliquot subjected to purification of 11.7%.

### Example 8

Purification of B-Phycoerythrin (B-PE) from Aqueous Extracts of Porphyridium cruentum Biomass on PVDF Membrane

A crude phycobiliprotein extract was obtained by suspending the freeze-dried P. cruentum biomass in an aqueous solution of NaCl 100 mM (120 mg of powder in 15 mL of solution). The suspension was kept at 4° C. for 24 hours, then centrifuged for 45 minutes (12,000 rcf, temperature=10° C.). The supernatant containing B-PE (hereinafter “crude extract”) was recovered and stored at 4° C.

The B-PE purification process was carried out by using a syringe as the chromatographic device and filters for syringes with membranes made of hydrophilic PVDF having a porosity 0.45 μm (Durapore®, average pore size 0.45 μm, Ø 33 mm, code SLHV033RS) or 0.22 μm (Durapore®, average pore size 0.22 μm, Ø 33 mm, code SLGV033RS) and applying the purification process described in the following. No cleaning cycle was carried out, while the purification cycle was performed as follows: an aqueous solution of NaCl 100 mM and ammonium sulfate 3 M were added to a 0.3 mL aliquot of crude extract with a B-PE content of about 0.1 mg, so as to have a final volume of 1.15 M ammonium sulfate solution equal to 3 mL. The filter was washed with 5 mL of deionized water and then conditioned with 3 mL of ammonium sulfate 1.15 M. The extract solution in ammonium sulfate 1.15 M was loaded onto a PVDF filter with 0.22 μm porosity, and in parallel on a filter, always made of PVDF, with medium-sized pores 0.45 μm, conditioned with ammonium sulfate 1.15 M. After filtration, the retentate on the membrane was washed with 3 mL of aqueous ammonium sulfate solution 1.15 M, recovered with 3 mL of NaCl 100 mM and its degree of purity was spectrophotometrically determined.

In both cases, B-phycoerythrin (B-PE) was obtained, having a degree of purity of about 3.95 and a yield of approximately 50%.

### Example 9

Purification of B-Phycoerythrin (B-PE) from Aqueous Extracts of Porphyridium cruentum Biomass on PVDF Membrane

A crude phycobiliprotein extract was obtained by suspending 170 mg of freeze-dried P. cruentum biomass in powder in 21 mL of saline phosphate buffer (PBS, sodium phosphate 10 mM, NaCl 100 mM) at pH 7. The suspension was maintained at 4° C. for 24 hours, then centrifuged for 45 minutes (12,000 rcf, temperature=10° C.). The supernatant containing B-PE (hereinafter “crude extract”) was recovered and stored at 4° C.

The B-PE chromatographic purification process was carried out using a glass vacuum device for microfiltration assembled with a hydrophilic PVDF membrane (Durapore®, average pore size 0.45 μm, diameter 47 mm, code HVLP 04700) and carrying out the cleaning and purification cycles as follows:
cleaning cycle: the membrane was washed with 100 mL of deionized water and then conditioned with 5 mL of an aqueous solution of ammonium sulfate 0.83 M. PBS at pH 7 and an aqueous solution of ammonium sulfate 3 M were added to an aliquot of 2 mL of crude extract with B-PE content equal to about 0.7 mg, so as to have a final volume of 9 mL of extract in a solution of ammonium sulfate 0.83 M. The solution was loaded on the membrane and filtered; the permeate was collected in a 100 mL clean tailed flask. The volume of the solution was carefully measured and, if necessary, it was brought back to 9 mL using an aqueous solution of ammonium sulfate 0.83 M.
purification cycle: to the permeate in aqueous solution of ammonium sulfate 0.83 M obtained from previous cleaning cycle, an aqueous solution of ammonium sulfate 3 M was added so as to obtain a solution of phycobiliproteins having a concentration of ammonium sulfate 1.224 M. This solution was loaded onto a new PVDF membrane, using the usual procedure: the membrane was washed with deionized water (100 mL) and then conditioned with 5 mL of an aqueous solution of ammonium sulfate having the same ammonium sulfate concentration as the solution of phycobiliproteins (1.224 M). The phycobiliprotein solution in ammonium sulfate 1.224 M was loaded onto the membrane and filtered. The retentate bound onto the membrane was washed with 10 mL of an aqueous solution of ammonium sulfate 1.224 M, then desorbed with 10 mL of PBS at pH 7, recovered in a 100 mL clean tailed flask and its degree of purity was spectrophotometrically determined.

B-phycoerythrin (B-PE) was obtained with a purity degree of 4.0, and with a yield with respect to the B-PE content in the crude extract aliquot, subjected to purification, of 38.0%.

The present invention has been so far described with reference to its preferred embodiments. It is to be understood that there may be other embodiments which refer to the same inventive core, all falling within the scope of protection of the claims set forth in the following.

### BIBLIOGRAPHIC REFERENCES

[1] P. Yu, Y. Wu, G. Wang, T. Jia, Y. Zhang, Purification and bioactivities of phycocyanin, Crit. Rev. Food Sci. Nutr. 57 (2017) 3840-3849. DOI: 10.1080/10408398.2016.1167668;
[2] Q. Liu, Y. Huang, R. Zhang, T. Cai, Y. Cai, Medical application of Spirulina platensis derived C-phycocyanin, Evid.-based Complement. Altern. Med. 2016 (2016). DOI: 10.1155/2016/7803846;
[3] G. Patil, K. S. M. S. Raghavarao, Aqueous two phase extraction for purification of C-phycocyanin, Biochem. Eng. J. 34 (2007) 156-164. DOI: 10.1016/j.bej.2006.11.026;
[4] R. Chaiklahan, N. Chirasuwan, V. Loha, S. Tia, B. Bunnag, Separation and purification of phycocyanin from Spirulina sp. using a membrane process, Bioresour. Technol. 102 (2011) 7159-7164. DOI: 10.1016/j.biortech.2011.04.067;
[5] K. Minkova, M. Tchorbadjieva, A. Tchernov, M. Stojanova, L. Gigova, M. Busheva, Improved procedure for separation and purification of Arthronema africanum phycobiliproteins, Biotechnol. Lett. 29 (2007) 647. DOI 10.1007/s10529-006-9274-5;
[6] B. Soni, U. Trivedi, D. Madamwar, A novel method of single step hydrophobic interaction chromatography for purification of phycocyanin from Phormidium fragile and its characterization for antioxidant property, Bioresour. Technol. 99 (2008) 188-194. DOI: 10.1016/j.biortech.2006.11.010;
[7] G. Patil, S. Chethana, M. C. Madhusudhan, K. S. M. S. Raghavarao, Fractionation and purification of the phycobiliproteins from Spirulina platensis, Bioresour. Technol. 99 (2008) 7393-7396. DOI: 10.1016/j.biortech.2008.01.028;
[8] S. Muthukumar, T. Muralikrishnan, R. Mendhe, A. S. Rathore, Economic benefits of membrane chromatography versus packed column purification of therapeutic proteins expressed in microbial and mammalian hosts, J. Chem. Technol. Biot. 92 (2017) 59-68. DOI: 10.1002/jctb.5064;
[9] Z. Liu, S. R. Wickramasinghe, X. Qian, Membrane chromatography for protein purifications from ligand design to functionalization, Sep. Sci. Technol. 52 (2017) 299-319. DOI: 10.1080/01496395.2016.1223133.

1. A process for the separation and purification of phycobiliproteins starting from crude extracts of cyanobacteria and/or algae containing them, comprising a purification cycle of membrane chromatography comprising:
i) passing a saline aqueous solution of said crude extracts on a porous hydrophilic membrane, having low binding affinity for proteins, as the stationary phase, wherein said saline aqueous solution of crude extracts of phycobiliproteins is an aqueous solution of a salt S having a concentration [S]₁ that is a first mobile phase;
ii) desorption of a retentate bound to said membrane with a solvent that is a second mobile phase and is selected from the group consisting of water, an aqueous solution of said salt S at a concentration [S]₂<[S]₁, and an aqueous solution of a salt S′ that is a chaotropic agent stronger than said salt S.

2. The process according to claim 1, further comprising, upstream of said purification cycle, a chromatographic cleaning cycle on said porous hydrophilic membrane of said crude extracts in an aqueous solution of said salt S at concentration [S]₀ that is a third mobile phase, lower than said concentration [S]₁ of said salt S in the step i).

3. The process according to claim 1, wherein said porous hydrophilic membrane is a hydrophilic membrane in PVDF (polyvinylidene fluoride) or in polycarbonate.

4. The process according to claim 3, wherein said porous hydrophilic membrane is a hydrophilic membrane in PVDF.

5. The process according to claim 1, comprising more purification cycles carried out consecutively at the same or increasing concentrations of the saline aqueous solution of said salt S by passage on said porous hydrophilic membrane of the permeate obtained from the preceding cycle up to the separation of the desired phycobiliprotein having the desired purity degree.

6. The process according to claim 1, wherein said porous hydrophilic membrane, before each said purification cycle and/or before said cleaning cycle, is previously treated with a saline aqueous solution of said salt S at concentration [S]ₙ, wherein [S]ₙ is the concentration of the salt S in the cleaning step or in the nth cycle of purification.

7. The process according to claim 1, wherein said crude extracts are extracts of cyanobacteria or algae selected from extracts of Arthrospira platensis (Spirulina) and extracts of Porphyridium cruentum.

8. The process according to claim 1, wherein said salt S is ammonium sulfate.

9. The process according to claim 1, wherein said aqueous solution of salt S′ is selected from aqueous solutions of sodium chloride and saline phosphate buffer.
